(19) United States
(12) Patent Application Publication
(10) Pub. No.: US 2018/0144832 A1
(43) Pub. Date: May 24, 2018

(54) REAL-TIME OR JUST-IN-TIME ONLINE ASSISTANCE FOR INDIVIDUALS TO HELP THEM IN ACHIEVING PERSONALIZED HEALTH GOALS

(71) Applicants: ANAND SUBRA, Plymouth, MN (US); NARAYANAN RAMASUBRAMANIAN, Fremont, CA (US)

(72) Inventors: ANAND SUBRA, Plymouth, MN (US); NARAYANAN RAMASUBRAMANIAN, Fremont, CA (US)

(21) Appl. No.: 15/875,771

(22) Filed: Jan. 19, 2018

Related U.S. Application Data
(62) Division of application No. 15/467,341, filed on Mar. 23, 2017.
(60) Provisional application No. 62/312,649, filed on Mar. 24, 2016.

(51) Int. Cl. G16H 80/00; H04L 29/08; G09B 19/00; G06T 11/60; G06T 11/00; G16H 10/60; G06Q 10/10; G06N 3/00; H04W 4/30; G06Q 30/02

(52) U.S. Cl. CPC G16H 80/00; H04L 67/306; G09B 19/0092; G06T 11/60; G06Q 30/0267; G16H 10/60; G06Q 10/109; G06N 3/004; H04W 4/30; G06T 11/001

(57) ABSTRACT

A method and a system for providing real-time assistance to users in achieving their personalized health goals through a mobile phone with an integrated software application by uploading the photographs to a secured database of the software application of the mobile phone provided with specific text comments or requests placed in appropriately classified input queues for assigning to a qualified nutritionist or an Artificial Intelligence (AI) Program for analyzing the uploaded photographs and generating specific modifications to the food items on the photograph of a meal by applying the user-specific weight-loss/meal modification rules, displaying the analysis information and the specific modifications on the food items to the user on the mobile phone screen. The system and method also comprises a container, a partition plate, a closable lid for storing the food items that are rejected by the nutritionist or the AI and a placemat printed with colored squares patterns.

### FIELD OF INVENTION

The present invention relates to providing an individual a real-time or an online assistance in achieving personalized health and wellness goals such as weight loss, adherence to various types of diets, prevention of heart disease or cancer, proper medication-taking, etc. Further, the present invention provides a smart-phone application with facilities for individuals to upload a picture of a meal they are about to consume and receive a real-time or just-in-time modification to the meal generated by a qualified nutritionist or an artificial intelligence (AI) program. Specifically, the present invention eliminates the need for individuals to remember dietary plans or guidelines or analyze any detailed information about the meal, and makes it very easy for individuals to maintain health.

### BACKGROUND

The burden of being overweight or obese, in terms of health problems and expenditures, is well known. Numerous weight loss approaches exist that require substantial changes to diets and exercise routines, sustained tracking and detailed numeric analysis of ingredients, caloric values, fat content, etc. Individuals are unable to adhere to and maintain these requirements, which typically leads to temporary weight loss, but the lost weight is regained, sometimes repeatedly.

Weight loss approaches based on diet plans require individuals to first remember to follow the plans, and second, to do a good job of actually following the plans. Individuals often have difficulty remembering their respective list of foods to avoid or reduce. Tools are available to analyze the content of foods, and many individuals try to adjust their eating to comply with their respective diet plans.

Users packed with busy work schedules and under social pressure to join others in the act of eating find it very difficult to sustain over time. Pre-packaged diets packages help in this regard, but they are expensive and individuals can get tired of eating the same items over and over, which leads to non-adherence. Further, deprivation of certain favorite food items results in frustration and can lead to abandonment. Other approaches provide detailed information regarding the calories, fat, sodium, etc., content of foods, and rely on the diligence of the individual to check, analyze and modify their eating habits in order to stay within their dietary plans or guidelines. Again, adherence eventually breaks down because of the amount of detailed effort involved, and only the most diligent individuals continue for the longer term.

Many systems or methods or devices have been introduced globally to adopt good dietary habits to build healthier lifestyle. Since it has become impractical for most individuals to exercise for more than an hour or two a day, modifying their food intake is more effective than exercise.

U.S. Pat. No. 6,478,736 discloses a health management system for a person, in which the person's resting metabolic rate (RMR) is determined at intervals using an indirect calorimeter. RMR values are used in setting and revising goals in, for example, a weight control program. The effects of a weight control program on RMR can hence be compensated for, which enables an improved weight control program to be developed. In one embodiment, the person is provided with a portable electronic device, for use as a caloric intake calculator, caloric expenditure calculator, and caloric balance calculator. This user needs to carry the system whenever he or she wishes to consume food, which is not feasible all the time.

U.S. Pat. No. 7,959,567 relates to an apparatus for detecting at least one of human physiological and contextual information from the body of a wearer that includes a sensor device adapted to be worn on the body having one or more sensors selected from the group consisting of physiological sensors and contextual sensors and an I/O device in electronic communication with said sensor device. The I/O device includes means for displaying information and a dial, the dial being supported for rotational movement about an external surface of the I/O device. The dial enables the wearer to enter information into the I/O device. The I/O device may further include at least one button that also enables the wearer to enter information into the I/O device. The task of entering information relating to type and quantity of meals sometimes results in inaccurate estimation of caloric content if a user is not sure about the relative size of the meal.

U.S. Pat. No. 5,454,721 discloses a system intended to teach individuals the relationship between the visual size and a few nutritional characteristics of portions of food by using either a life size image of, or the corporeal finger of the individual, as a scale against images of different sized portions of different kinds of food, while showing a few nutritional characteristics of such portions. The system proposed is minimal when compared to the present invention's features. This system does not evaluate the user's ability to visually estimate macro and micronutrient content of meals nor does it permit analysis of an individual's dietary pro-activities.

US20070179359 discloses a receiving a caloric request and a resting metabolic rate, computing an expended number of calories based on the user's resting metabolic rate and physical activity performed by the user, computing a consumed number of calories based on food the user consumes, determining a status for the user based on whether the user is to consume calories or expend calories, and sending an alert to the user. This method calculates the estimated calories based on the physical activity performed by the user and resting metabolic rate. Performing physical activity is not feasible for every user at every time with respect to their busy schedule.

However, these techniques can sometimes be difficult to employ. As an example, during a busy day, people may forget to exercise or count caloric intake. As another example, people who are traveling may be unable to easily locate activity centers or food sources that help them to manage their health. Often, people lack the motivation to live healthy lives.

The advent of smart phones with built-in cameras makes it possible to provide real-time assistance in the form of suggestions or comments on meals about to be consumed. Therefore, there is a need to provide a method and system operable by a software application integrated with a smart mobile phone to maintain a healthy lifestyle by a user attaining real-time assistance in estimating nutritional attributes in a meal, modifying the meal and encouraging them to maintain health for a longer duration.

### SUMMARY OF THE INVENTION

The invention comprises a method for providing real-time assistance to users in achieving their personalized health goals through a mobile phone comprising steps of:
installing a software application in the user's mobile phone;
registering with the said software application by providing personalized profile parameters, personalized health goals, other health goals of a user;
providing a secured database to store photographs of food items/meals to be consumed by a user;
analyzing the profile parameters of each user and generating specific ranked weight-loss rules/meal modification rules applicable to that particular user;
generating specific modifications to the food items on the photograph of a meal by applying the user-specific weight-loss/meal modification rules;
displaying the analysis information and the specific modifications on the food items to the user on the mobile phone screen;
tracking the status/modification stage of the image by providing a speed indicator, and
displaying a timeline of previously uploaded photographs of meals consumed, modifications, and other information, along with the means to rate the quality of the modifications and the level of adherence by the user.

A feature of this invention is to provide real-time assistance to users in achieving their personalized health goals through a mobile phone, wherein the registered user submits specific information for analyzing.

Another feature of this invention is to provide real-time assistance to users in achieving their personalized health goals through a mobile phone, wherein the registered user submits specific information for analyzing the uploaded photographs of the food items by a nutritionist/Artificial Intelligence (AI) for providing suggested modifications.

Another feature of this invention is to provide real-time assistance to users in achieving their personalized health goals through a mobile phone, wherein the registered user submits specific information for analyzing the uploaded photographs of the food items by a nutritionist/Artificial Intelligence (AI) and providing adherence/evaluation by the registered user.

Another feature of this invention is to provide real-time assistance to users in achieving their personalized health goals through a mobile phone, wherein the registered user obtains assistance in neutralizing a food craving.

Yet another feature of this invention is to provide real-time assistance to users in achieving their personalized health goals through a mobile phone, wherein the registered user submits specific information for analyzing the uploaded photographs of the unconsumed food items stored in a container or a device or a plate.

A further feature of this invention comprises a system for providing real-time assistance to users in achieving their personalized health goals through a mobile phone comprising of:
a mobile phone integrated with a software application;
one or more secured databases to store the photographs of the food items/meals, generated ranked weight-loss rules/meal modification rules, suggested modifications;
a processor for performing analysis of nutritional information;
wherein comprises a container or a device provided with a closable lid and a knob for categorizing the food items for consuming placed on a plate,
wherein creates a partition of the plate into which the rejected food items are placed using the device, and
wherein comprises a placemat printed with colored squares of standard size for providing size reference for estimating size and/or quantity of food items on the plate that is placed on top of the placemat.

Another feature of this system is to generate reports depicting past trends, current status and future predictions and displays the reports at the time of analyzing.

### BRIEF DESCRIPTION OF DRAWINGS

FIG. 1a: illustrates the process flow for downloading and registering with the mobile phone application according to the preferred embodiment.

FIG. 1b: illustrates the process flow for generation of user-specific ranked weight-loss rules according to the preferred embodiment.

FIG. 2: illustrates the process flow for sending a meal-time reminder to the registered user according to the preferred embodiment.

FIG. 3: illustrates the process flow for storing the uploaded photographs in a secured database and queuing of the photographs according to the preferred embodiment.

FIG. 4a: illustrates the process flow for nutritionists to access the system for providing modifications according to the preferred embodiment.

FIG. 4b: illustrates the process flow for the AI Program to provide modifications according to the preferred embodiment.

FIG. 5: illustrates the process flow for notifying the users regarding the suggested modifications and sending the appropriate data according to the preferred embodiment.

FIG. 6: illustrates the process flow for escalating exceptions for further handling by experts according to the preferred embodiment.

FIG. 7: illustrates the process flow for viewing a timeline of their past meal photographs, modifications and comments and indicating their adherence to the modifications according to the preferred embodiment.

FIG. 8: illustrates the process flow for setting up ‘Help Groups’ for assistance according to the preferred embodiment.

FIG. 9: illustrates the process flow assisting the ‘Help Groups’ dealing with food cravings at any time according to the preferred embodiment.

FIG. 10: illustrates the process flow for requesting assistance from a nutritionist, dietician or other professional at any time according to the preferred embodiment.

FIG. 11: illustrates the process flow for calculating the modification quality ratings by nutritionist and user's adherence to it according to the preferred embodiment.

FIG. 12a-b: illustrates a container into which rejected food items as part of the nutritionist's modification are placed according to the preferred embodiment.

FIG. 12c: illustrates a device for creating a partition on a plate into which rejected food items are placed according to the preferred embodiment.

FIG. 12d: illustrates a pouch-like device into which rejected food items are placed according to the preferred embodiment.

FIG. 13a: illustrates a processed image of a plate with food items that have been color-coded based on their dominant nutritional attribute value according to the preferred embodiment.

FIG. 13b: illustrates a processed image of a plate with food items that are superimposed, pie-charts or other like representations indicating the relative proportions of various nutritional attributes of each food item according to the preferred embodiment.

FIG. 14a: illustrates a placemat printed with grey and white squares of one-inch size to provide a sizing reference with the plate placed on top of the placemat according to the preferred embodiment.

FIG. 14b: illustrates a placemat printed with colored squares of one-inch size to provide color and sizing reference with the plate placed on top of the placemat according to the preferred embodiment.

FIG. 15: illustrates a plate partition as shown in FIG. 12c printed with standard sized colored squares that serve as reference in identifying and estimating the food items on the plate according to the preferred embodiment.

FIG. 16: illustrates the process flow for estimating the nutritional values of the food items for either a plate of food or the food items set aside as the modification and report generation according to the preferred embodiment.

FIG. 17: illustrates the process flow for predicting a user's craving times and responding to it according to the preferred embodiment.

FIG. 18: illustrates the process flow for monitoring the user's eating patterns and flag issues raised by the user according to the preferred embodiment.

FIG. 19: illustrates the process flow for providing coaching to a user through two-way rich media according to the preferred embodiment.

### DETAILED DESCRIPTION OF THE PREFERRED EMBODIMENTS

A preferred embodiment of the present invention addresses the needs of individuals desiring to lose weight by modifying their food intake by analyzing the content of foods and assisting the individuals to adjust their eating to comply with their respective diet plans.

The preferred embodiment provides a smart mobile phone application provided with facilities for individuals to upload a picture of a plate of food items or meals they are about to consume and receive a real-time or just-in-time modification to the meal generated by a nutritionist or an artificial intelligence (AI) program. The modification is generated based on the individual's personalized profile parameters, personalized weight-loss goals and the meal to be consumed as well as the history of meals previously consumed. Since the modification is generated at the point of consumption, the individual does not need to remember any dietary plans or guidelines or analyze any detailed information about the meal in order to make practical decisions about what to eat. The individual uploads a picture of the meal, receives a real time modification and eats the meal according to the modification.

In the mobile application, individuals register as users and enter certain required profile parameters, and use the smart-phone camera to take photographs of meals and upload them to a secured database. Authorized nutritionists, dieticians or professionals examine the uploaded photographs and related information and compose specific modifications to the meals based on the individuals' respective profile parameters and their personalized weight-loss goals. The user receives a customized modification to the uploaded photograph by a qualified nutritionist. This greatly simplifies the weight-loss regimen.

Nutritionists or dieticians edit or add pre-configured clarifying text, graphics, audio or video to the uploaded photographs in order to indicate the specific modifications. This embodiment also envisions the use of artificial intelligence techniques to algorithmically select uploaded photographs, apply user-specific weight-loss rules, generate and compose suitable modifications for the associated users. In cases where the photographs are not readable, the nutritionist or the artificial intelligence program marks them as exceptions and sets up a queue for further handling by more skilled human experts.

Once the modifications are composed, push notifications are automatically sent to the respective users to view the modifications. The time of elapse of tracking for each photograph is monitored, and if it is elapsed beyond certain threshold limits delay notifications, tips and other information are automatically sent.

The mobile application also displays a timeline of past photographs, modifications and comments, so the user may scroll back and forth to examine them at any time, zoom in to a specific past photograph, indicate actual adherence to the modifications and rate their quality, timeliness and effectiveness.

The preferred embodiment also enables users to get answers to weight-loss or other health-related questions at any time by initiating a help request and directing it to a nutritionist or a dietician or other health professional, or an artificial intelligence program. Also, the users to get assistance in dealing with food cravings at any time by initiating a craving help request to assigned helper or friend groups, who may respond and attempt to distract the requesting user.

A further provision aggregates, for a particular user, the nutritional values of the food ingested on a particular day, and suggest what items may be eaten at the end of the day to ensure that the user's specific daily dietary limits are not exceeded. This also provides information regarding nearby restaurant menus or grocery store items in the vicinity of the user's current location and suggests consuming the food items available in the restaurant menus or grocery stores.

The present invention also provides a separate container or plate separator device for placing food items that are flagged as ‘do not eat at this meal’ or rejected as part of the nutritionist's modification, for ingestion by the user, a different person or animal, or for disposal, at a later time. In addition to this, the present invention also provides a placemat printed with grey and white or color patterned squares of standard size to provide a sizing and color reference, as assistance for estimating the nature, size and/or quantity of food items on a plate that is placed on top of the placemat.

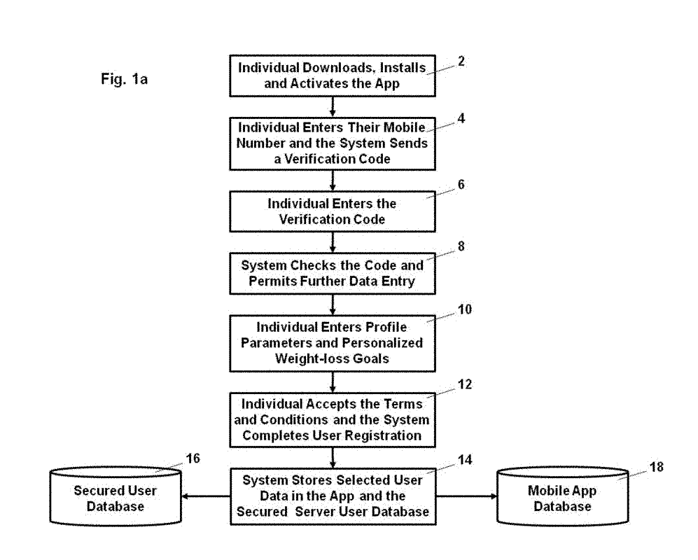

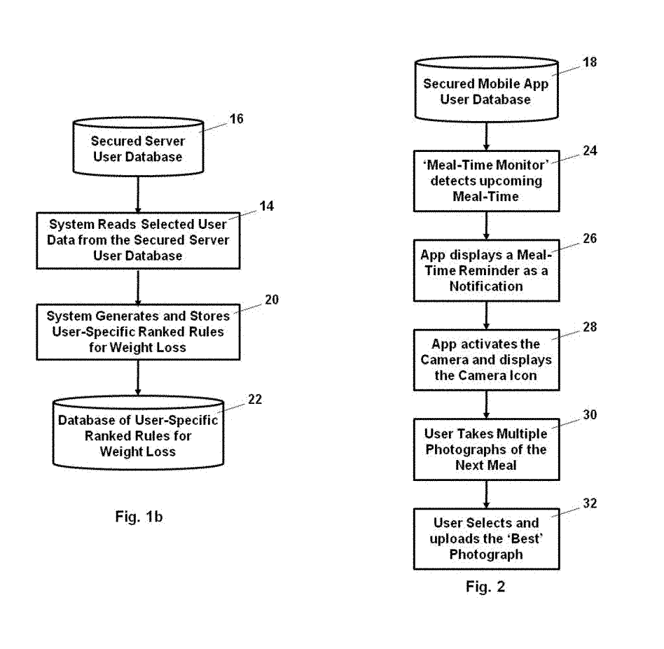

Referring now to FIG. 1a, illustrates a process flow for downloading and registering with the mobile phone application by an individual user. An individual can locate and download the mobile application from play store or from a website and to activate the application by installing in the mobile phone [2]. The application prompts the individual to enter the mobile phone number and receives a verification code and sends an authentication code as one-time-password to the mobile phone number for verification. The application then allows the user to enter the one-time-password [6]. The system verifies the entered code and permits the user for further data entry [8] that includes personalized profile parameters, personalized health goal, other health goals, personalized weight-loss goals, etc. [10]. The user's personalized profile parameters include age, gender, body shape, current weight & height, target weight, blood pressure, cholesterol, blood sugar, etc. The personalized health goals include target amount of weight to reduce, target amount of blood pressure or blood sugar to be attained, etc.

Further, the user then accepts the terms and conditions of the system to attain the assistance of the application and completes the registration [12]. The system stores the selected user data in the mobile phone application and in the secured server [14] with respective databases as secured server user database [16] and secured mobile app user database [18]. Where, the user can be a person or a parent or a caregiver or a pet-owner seeking real-time assistance.

FIG. 1b, illustrates the process flow for generation of user-specific ranked weight-loss rules based on user-profile parameters and other user data from the secured server database. The system reads the user data and parameters [14] from the secured server user database [16]. These user personalized profile parameters and other parameters such as recent meal history serve as inputs to the algorithms for generating the user specific ranked weight-loss rules. The ranked weight-loss rules also include meal modification rules that are generated by various algorithms for different health goals that are assigned based on the specific user's profile data and/or health goals. The system generates the user specific ranked rules for weight-loss [20] and stores the generated rules in a database of user specific ranked rules for weight loss [22].

FIG. 2, illustrates the process flow for sending a meal-time reminder to the registered user by prompting the user to activate the mobile application. The application runs a typical ‘Meal-Time Monitor’ routine [24] in the background that detects the occurrence of a ‘typical meal-time’, based on past history of meals and other relevant data entered by a registered user that is stored in the secured mobile application user database [18]. When an impending meal-time is detected, the application displays a Meal-Time Reminder as a notification [26] that is displayed on the mobile phone screen. Simultaneously, the application also activates the camera and displays the camera icon for the user to capture a photograph of the next meal [28]. The users can also use 2D or a 3D camera affixed to their eyeglass or other means to capture photographs of the food item/meals to be consumed.

Further, the users are allowed to capture photographs of the food items at a grocery store to attain personalized instructions on their suitability, based on personalized health profile and other health goals. The registered user can also capture and upload additional or sequential photographs representing additional servings at a particular meal. The user captures multiple photographs of the meal [30], views them, and selects the best photograph with respect to clarity and uploads it [32] by clicking on the ‘upload’ button. Before uploading, the user is allowed to add specific graphical modifications or text comments or audio comments or requests and/or other related information to the captured photographs. These comments also include specific queries with respect to their weight-loss or health goals.

Further, means are provided for the registered users to add annotations by clicking or touching on the mobile phone screen on certain food items in the modified image to indicate respective food item names and to provide specific requests to the nutritionist. Additionally, means are provided for the registered user to seek advice from the nutritionist or AI on what to eat for dessert at the end of the meal, given the meal that has just been consumed and receive a response; on what to eat for dinner at the end of the day, given all the meals that have been consumed thus far and receive a response and to seek advice on food item or recipe swaps to improve the quality of their nutritional intake.

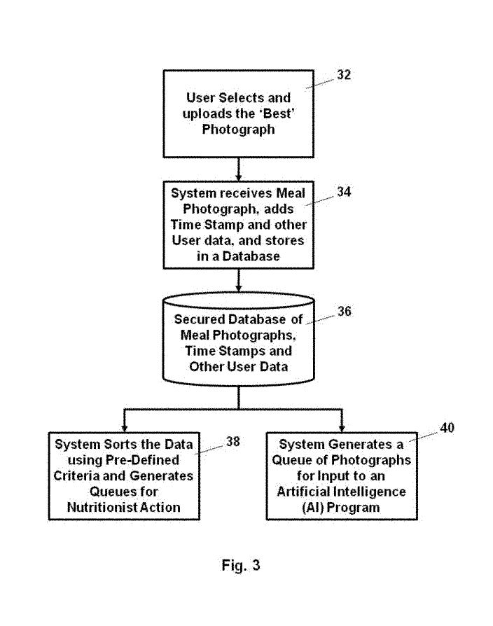

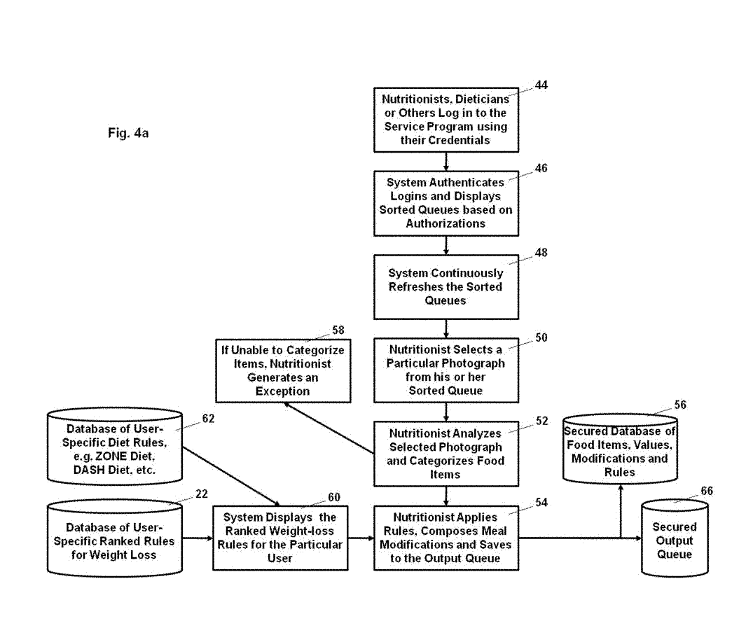

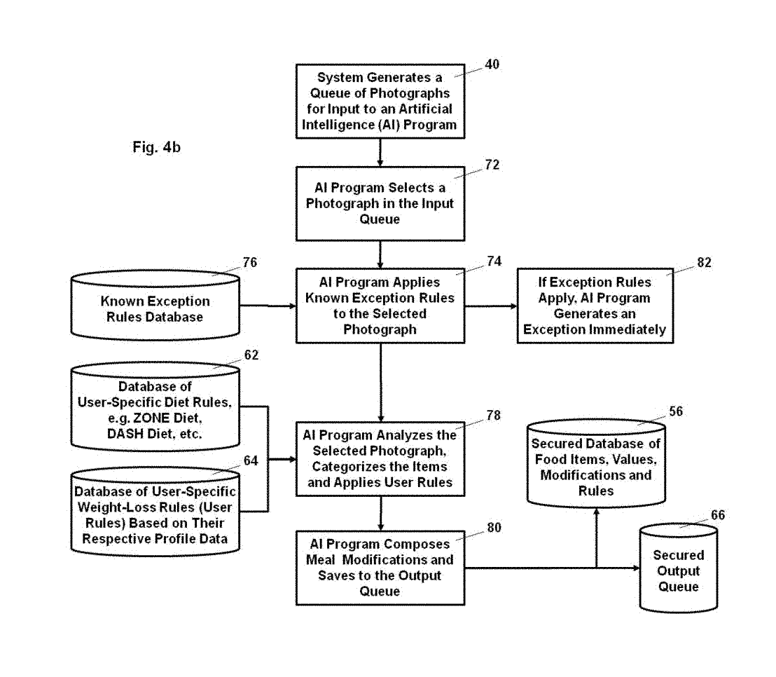

FIG. 3, illustrates the process flow for storing the uploaded photographs in a secured database and queuing of the photographs to initiate further action. After uploading the best photographs by the registered users, the uploaded photographs are received, identified, time-stamped &associated with other user data [34], and stored in a secured database of uploaded photographs. All the data received from multiple users is stored in a secured database of meal photographs, time stamps and other user data [36]. The system then sorts the data using pre-defined criteria and generates an input queue of photographs and associated data for further action by nutritionists, dieticians and other professionals [38]. The System also generates an input queue of photographs for input to an Artificial Intelligence (AI) Program [40] and the further process is depicted in FIG. 4b.

The input queues are classified based on the specific text comments or requests attached to the uploaded photographs for assigning to a particular nutritionist or an AI. This classification is attained by providing specific filters that include by user, by associated nutritionist, by artificial intelligence program or the like for assigning the uploaded photographs. Additional filters are also provided based on meal modification rules or other criteria to further classify the input queues, thereby presenting an input queue of uploaded photographs to which same rules or criteria are to be applied.

FIG. 4a illustrates the process flow for nutritionists to access the system for providing modifications on the uploaded photographs of the food items. Nutritionists log in to the system to view the input queues of uploaded photographs, compose and save meal modifications. In the customary manner, nutritionists, dieticians or other authorized professionals log in to the system [44] after their authentication. Further, the system displays sorted input queues of photographs and associated information based on their respective authorizations [46]. As new photographs are continuously uploaded by users, the system continuously refreshes the sorted input queues [48].

A particular nutritionist, dietician or professional with proper authorizations, who has accessed his or her respective sorted queue, views thumbnails of the uploaded photographs and associated data and selects a particular photograph [50]. The System then displays a larger version of the selected photograph along with the applicable Ranked Weight-loss Rules for the Particular User [60]. The rules are generated using the Database of User-Specific Diet Rules [62] and the Database of User-Specific Ranked Rules for Weight Loss [22] according to the present embodiment.

In other embodiments, the user-specific ranked rules may reflect other health objectives such as waist-reduction, heart-healthy, cancer-prevention, diabetes management, etc., or rules that enforce adherence to certain diets, e.g. DASH (Dietary Approaches to Stop Hypertension) Diet, etc. The Nutritionist analyzes the selected photograph and categorizes the food items in the meal [52]. The nutritionist then applies the user's respective weight-loss rules to compose specific Meal Modifications and Comments by using web-based applications or mobile phone applications or tablet-based applications, and saves those [54] to the Secured Output Queue [66]. The composed meal modifications and comments by the nutritionist include text based or graphic based comments or modifications on the photographs and also estimates of the nutritional attributes and their corresponding values of the food items or meals to be consumed or rejected by a user.

The text based comments include generic eating instructions, personalized eating instructions, pre-configured textual comments and textual indicators on the modifications to indicate the increase or decrease in the quantity of the food to be consumed. The graphic based comments include free-form line drawings, visual effects for increasing/decreasing the appearance of the food items, pre-configured clarifying graphics, pre-configured graphical indicators to indicate the increase or decrease in the quantity of the food to be consumed, magnification/de-magnification of specific food items indicating consumption levels. Means are provided for vocal comments and video comments to be attached. These comments are stored in the secured database as a data set associated with a specific image or from a specific registered user.

All these Photographs, modifications and associated data are also stored in the Secured Database of Food Items, Values, Modifications and Rules as an archive [56]. If the nutritionist is unable to recognize or categorize the food items in any photograph, then the nutritionist generates an exception for that particular photograph [58]. The nutritionist makes suggestions to reduce one item or to increase other items to compensate for nutritional values based on the specific user goals. Also, the nutritionist manually composes specific modifications to photographs based on the user's past meal history or user's specific dietary restrictions, profile parameters, personalized weight-loss goals or personalized diet plans.

In one embodiment, there exists a means on the mobile phone to view a suggested eating sequence for eating food items in the photograph which is automatically projected by the registered user by clicking on the said means include an icon or the like. This suggested eating sequence can be an unconventional sequence projected as a numbered list. The exception marked photographs [58] are reverted back to the respective registered user, thereby enabling the user to modify or add clarifying comments to that particular food item and re-upload the photograph.

In one embodiment, the nutritionist provides appropriate observations, or asks questions to a particular user depending on their diet; determines the consumable calories by a particular user based on the target amount of calories to be reduced, and determine the quantity of food items to be reduced/increased; uses pre-configured or free graphical/alphanumeric editing tools to indicate the food items to be modified and also sends encouraging or congratulatory comments when the plate of food needs no suggestions.

In all embodiments, the nutritionist may be a dietician or a veterinarian or other professional, providing assistance to individual adults, parents on behalf of children, adults on behalf of their parents, pet-owners and so on.

FIG. 4b illustrates the process flow for the AI Program to provide modifications on the uploaded photographs of the food items. The automated programs using artificial intelligence (AI) techniques access input queues of uploaded photographs and associated data, select a photograph, access the associated data and user-specific weight-loss rules to generate and compose modifications and comments. The AI program selects a photograph in the input queue [72] to apply known exception rules from the database Known Exception Rules Database [76] on the selected photograph [74]. If exception rules apply, the AI Program generates an exception immediately [82] and assigning an exception mark to that particular image if unrecognizable or not clear to be properly analyzed or if the program is unable to generate modifications. The AI program sets up an exception queue for queuing of photographs marked with exceptions and automatically send notifications to an expert panel or agent for further processing and selects the next photograph from the input queue.

The AI program analyzes the selected photograph, categorizes the food Items and applies user rules [78] to compose meal modifications [80] and saves them to a Secured Output Queue [66]. All Photographs, modifications and associated data are also stored in the Secured Database of Food Items, Values, Modifications and Rules as an archive [56]. The user-specific diet and weight-loss rules [78] are generated using the database of User-Specific Diet Rules [62] and the database of user-specific weight-loss rules based on their respective profile data [64] stored in the database of User-Specific Ranked Weight Loss Rules [22]. The AI program also displays a timeline of past photographs, modifications and comments to the registered user for examination and sends automated reminders or notifications including tips related to health, weight-loss, meal-time reminders or the like.

In one embodiment, the AI program suggests modifications to reduce one item or to increase another item to compensate nutritional values based on specific user goals and overlays color shading within the food item borders to indicate each item's respective dominant nutritional attribute thereby assisting the nutritionist to recognize the items need to be modified. The AI program automatically edits the uploaded image by inserting text icons and graphics to convey the suggested modifications for approval by the nutritionist.

In one embodiment, the AI directs the nutritionist to estimate the nutritional attributes of the food items after identifying the respective food items, and by intimating the particular user's amount of nutritional attributes consumed on a particular day, enables the nutritionist to recommend the items to be eaten by the end of that day to maintain the user's specific daily dietary limits. In another embodiment, the AI determines the current physical location of a user from the mobile phone to provide information relating to restaurants, menu items, grocery stores, and other places where meals or food items are available in the vicinity of the user's current location and intimates a particular user the amount of nutritional attributes consumed on a particular day and recommends the items that are available in restaurants, grocery stores, etc., in the vicinity of the user's current location to be eaten by the end of that day to maintain the user's specific daily dietary limits. In yet another embodiment, the AI intimates the user on the amount of nutritional values to be consumed on a meal by meal, daily, weekly or other time period basis.

In one embodiment, the AI automatically generates and displays personalized/opening messages along with a calorie or other nutritional attribute value to be consumed for that day or to be consumed for lunch or to be consumed for dinner; automatically generates and displays personalized/opening messages with recommendations for the next meal as specific food items or recipe modifications or restaurant menu choices or products available in store shelves with modifications. In another embodiment, the AI monitors and analyzes the user's eating patterns and flags serious issues that prevent achievement of user objectives, and initiates or recommends a counseling session with an adviser or different AI program to provide personalized or general advice.

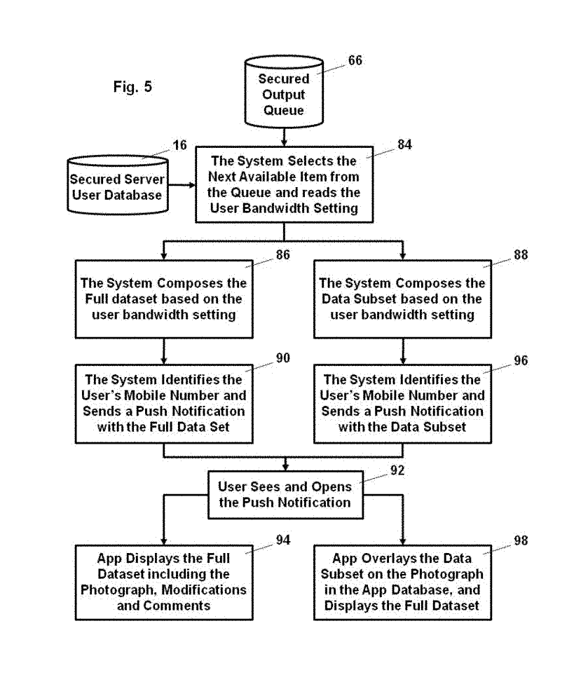

FIG. 5 illustrates the process flow for notifying the users regarding the suggested modifications and sending the appropriate data to them. The system notifies the users that a modification is ready to be viewed and enables the user to view the modification and comments. The System continuously scans the Secured Output Queue [66], Selects the next available item, reads the user bandwidth setting from the user profile parameters [84] stored in the Secured Server User Database [16]. Each image in the output queue is consolidated with the its respective identifiers, time-stamps, modifications, comments and other associated information to save the entire data set in the secured server database.

Based on the user bandwidth setting, the system either composes the full dataset [86], including the uploaded photograph, modifications, comments, and other associated information that is to be sent to the respective mobile phone, or the system composes a subset that excludes the uploaded photograph [88].The system then identifies the user's mobile number and sends a push notification with the full data set [90], or the data subset [96]. The sub-data set consists of modifications, comments and associated information, but not the image uploaded by the registered user. The user upon seeing the push notification opens it [92], and the mobile application automatically displays the full dataset, including the photograph, modifications and comments [94] or overlays the data subset on the photograph in the mobile application database, and then displays the photograph, modifications and comments [98].

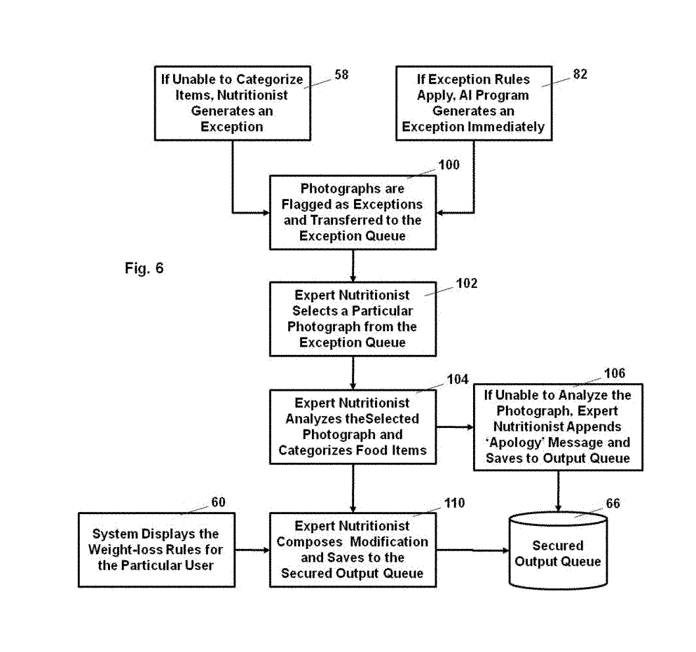

FIG. 6 illustrates the process flow for escalating exceptions for further handling by experts. An exception is manually generated by a nutritionist if he or she is unable to recognize or categorize items in an uploaded photograph [58]. Similarly, the AI Program generates an immediate exception if known exception rules applied to a particular uploaded photograph [82]. In both cases, the subject photographs and associated data are flagged as exceptions and transferred to an exception queue [100].

An expert nutritionist selects a particular photograph from the exception queue [102], analyzes the selected photograph and categorizes the food items [104] to compose the modification [110] by applying the weight-loss rules for the particular user [60] and save these modifications to the Secured Output Queue [66]. If the expert nutritionist is unable to analyze the Photograph, he or she appends an ‘Apology’ customer service type message [106] and saves it to the Secured Output Queue [66].

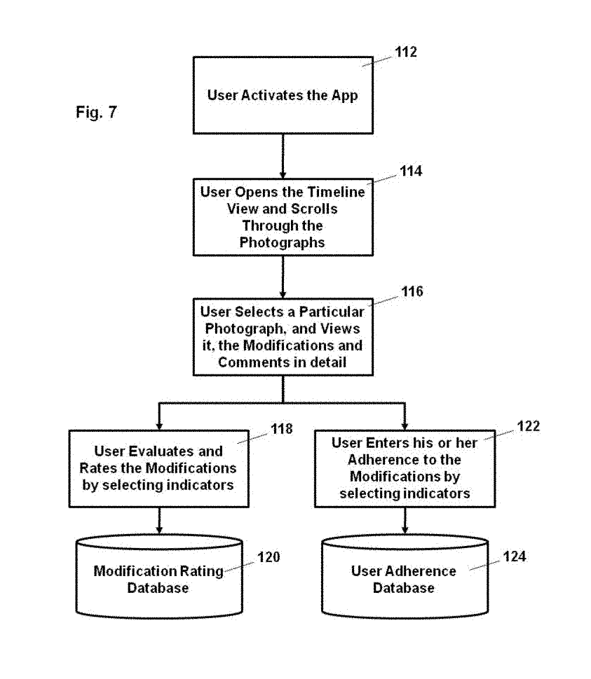

FIG. 7 illustrates the user process flow for viewing a timeline of their past meal photographs, modifications and comments and indicating their adherence to the modifications by providing rating on the quality of the modifications. The registered user activates the mobile application [112], opens the Timeline view and scrolls through the photographs [114]. The user then selects a particular photograph and views it, the modifications and comments in more detail [116]. In one embodiment, means are provided in the Timeline view for the user to enter health information such as weight, waist circumference, blood pressure, blood sugar, etc. manually/automatically by importing from various devices.

While viewing the photograph, modifications and comments, user may evaluate and rate the quality and effectiveness of the modifications [118], by selecting the appropriate graphical or other indicators provided in the display. The evaluation is also attained by providing comments, star-rating on a scale representing the quality of the modifications or by dragging on the timeline by holding a slider. The user rating is stored in the Modification Rating Database [120]. The user may also enter his or her adherence to the modifications by selecting the provided indicators [122], for example, whether he or she implemented the modifications fully or partially by uploading a second image depicting the actual post-consumption left-over's or unconsumed food items set aside. Adherence indications are stored in the User Adherence Database [124].

FIG. 8 illustrates the process flow for setting up ‘help groups’ for assistance by selecting individuals from their known contacts as well as from other users of the mobile application. The user may call upon any of the groups for assistance in dealing with a food craving, at any time of day. Any individual or individuals from the called-upon group may respond via any means available and try to distract or dissuade the user from succumbing to the craving. It is not necessary for ‘Help Friend’ Group members to be registered users of the application or services. The user activates the app in the usual manner [112], and uses the app function to set up at least one named ‘Help Friend’ groups [130]. Multiple such groups can be set up.

The user then selects certain personal contacts in order to invite them to join that particular group [132]. The system immediately sends Pre-Configured Text Message (SMS) invitation to the invitees [134], and resends the invitation one more time if any invitee does not respond after a set period [136]. If an invitee responds in the affirmative [138], then the system registers that invitee as a member of the user's named ‘Help Friend’ group [140]. If an invitee responds in the negative [142], the system does not register that invitee as a member of the user's named ‘Help Friend’ group and notifies the user that the invitation has been declined [144].

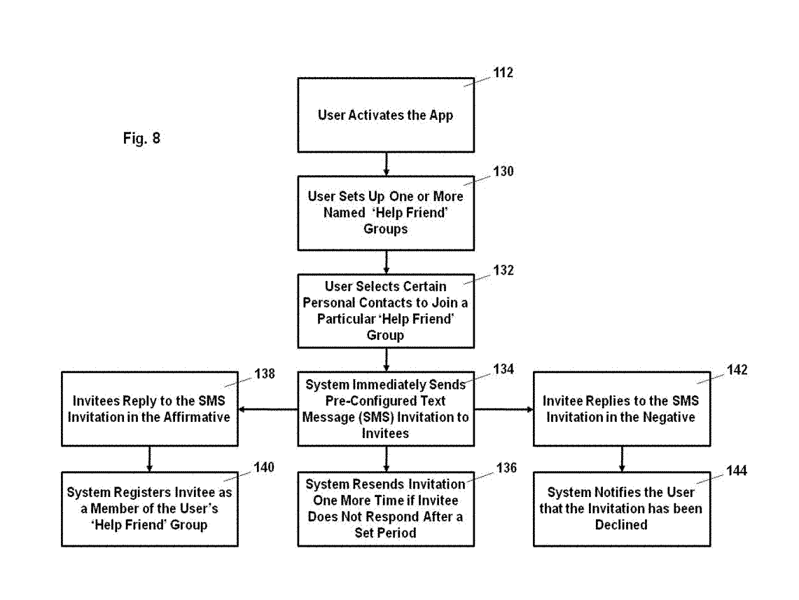

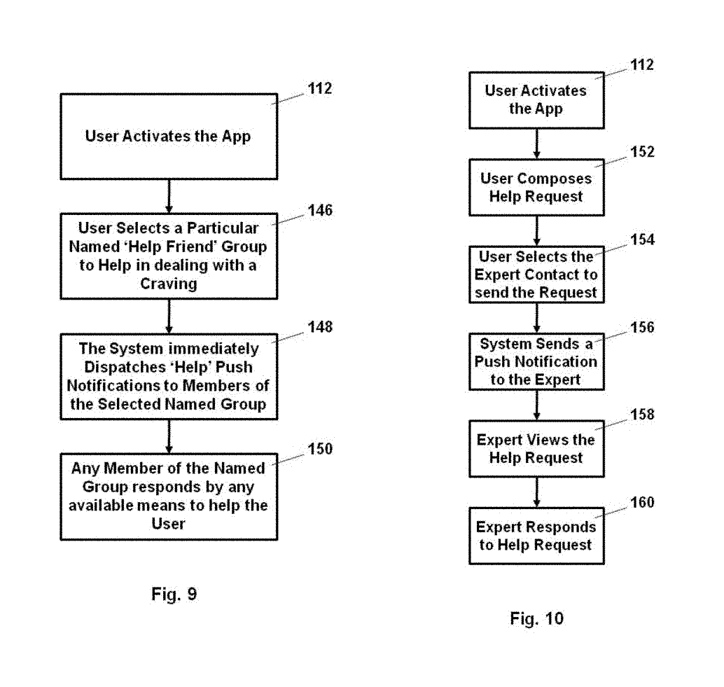

FIG. 9 illustrates the process flow assisting the ‘Help Groups’ dealing with food cravings at any time of the day. The user activates the app in the usual manner [112]. Assuming at least one named ‘Help Friend’ group with at least one contact has been set-up. The user can request assistance in dealing with a food craving at any time by selecting a particular named ‘Help Friend’ group [146]. The system immediately dispatches ‘Help’ push notifications to the members of the selected named group [148]. Any member of the named group, regardless of whether or not they are registered users, responds by any available means to help the user [150], including SMS (text messaging), chats, phone call, sending information (e.g. jokes, cartoons, videos, links, etc.) and attempts to distract the requesting registered user from the craving. Craving help requests are maintained open for a specified duration and are automatically closed after receiving at least one response, or at the end of the duration, whichever occurs first.

Alternatively, the AI automatically responds to the craving request to distract the registered user from the craving at typical snack-craving times or at anytime or if there is no response from any helper or group after a set duration. This AI automatically engages different friends when having different kinds of cravings by mapping specific cravings to a given friend's profile, sends a reminder or a notification to a specially-designated friend to proactively distract a specific registered user from a craving and analyzes patterns of craving to predict the next time of day when a registered user might get a craving and proactively suggests or engages a friend to respond to the craving.

FIG. 10 illustrates the process flow for requesting assistance from a helper, nutritionist, dietician or other professional (‘Expert’) at any time. The user may have a diet related question, may have taken a photograph of a meal and want a modification, or may want a modification to a recipe before cooking, or may be at a restaurant and want a menu item modification, etc. The user activates the app in the usual manner [112]. Assuming at least one ‘Expert’ with his or her contact information has been set up, the user can request help with any relevant subject matter as described above. The user composes a help request [152] by adding textual data and attaching photographs or other documents to the request. The user then selects at least one expert contact to send the request [154]. The system immediately sends a push notification to the selected expert [156]. The expert views the help request [158] and responds [160] by any available means to help the user, including SMS (text messaging), chats, phone call, or sending information (e.g. jokes, cartoons, videos, links, etc.), or suggest modifications using textual and graphical tools.

FIG. 11 illustrates the process flow for calculating the modification quality ratings by nutritionist and user's adherence to it and stored in their respective databases. The system reads the modification quality ratings in the modification rating database [120], sorts the data by nutritionist and calculates an overall quality rating that is, for example, an average of user ratings for a particular nutritionist over a set time period [162]. It then stores the nutritionist-respective ratings in the database of modification quality ratings by nutritionist [164]. Similarly, the system reads the user adherence database [124] sorts the data by user and calculates the user adherence indicator for each user [166]. It then stores the user-respective indicators in the database of user adherence to modifications by user [168].

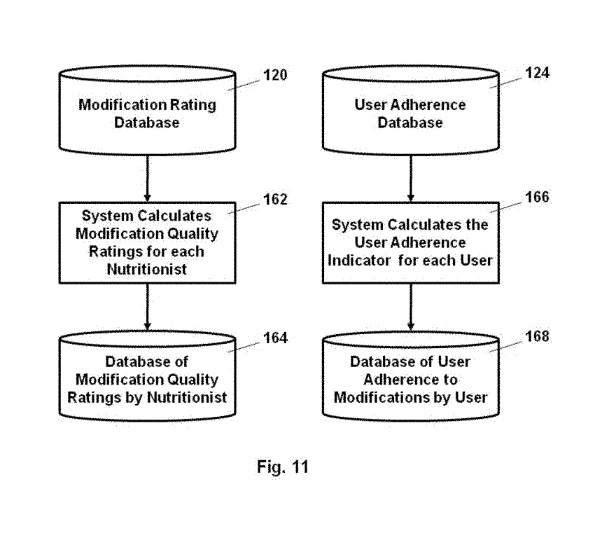

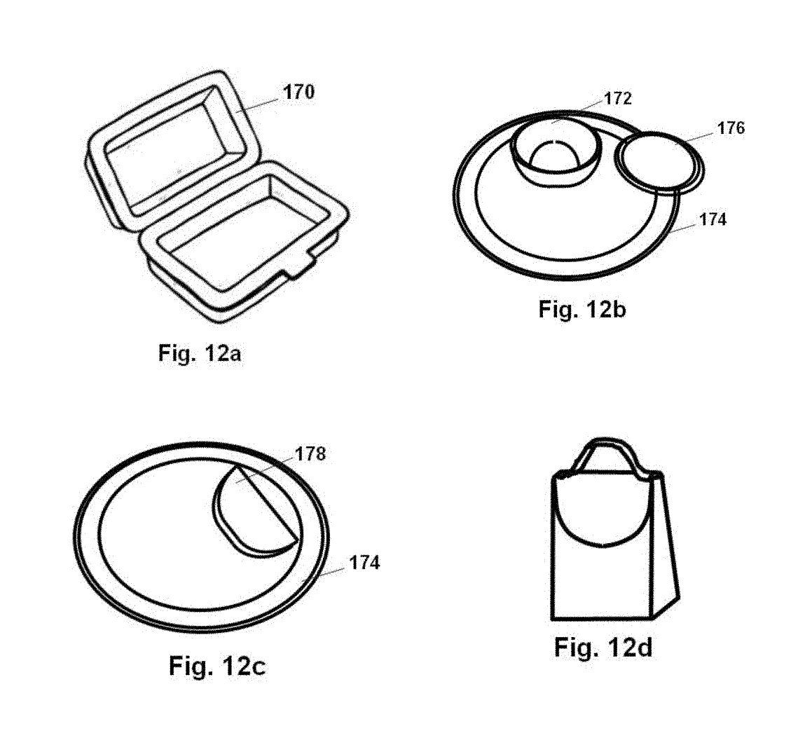

FIG. 12a-b illustrates a container into which rejected food items as part of the nutritionist's modification are placed. The container [170] accommodates food items that are flagged as ‘not for eating at this meal’ representing rejected food items as part of the nutritionist modification, for disposal or ingestion at a later time. In one embodiment, the container [172] is inbuilt into a plate [174] equipped with a separate lid [176] for accommodating food items that are flagged as ‘not for eating at this meal’ representing rejected as part of the nutritionist modification as shown in FIG. 12b.

FIG. 12c illustrates a device for creating a partition on the plate into which rejected food items are placed. The device [178] that creates a partition on the plate [174] into which food items flagged as ‘not for eating at this meal’, representing rejected as part of the nutritionist's modification for consumption or disposal at later stage.

FIG. 12d illustrates a pouch-like device into which rejected food items are placed. The pouch-like device into which food items flagged as ‘not for eating at this meal’ as part of the nutritionist's modification for consumption or disposal at a later stage.

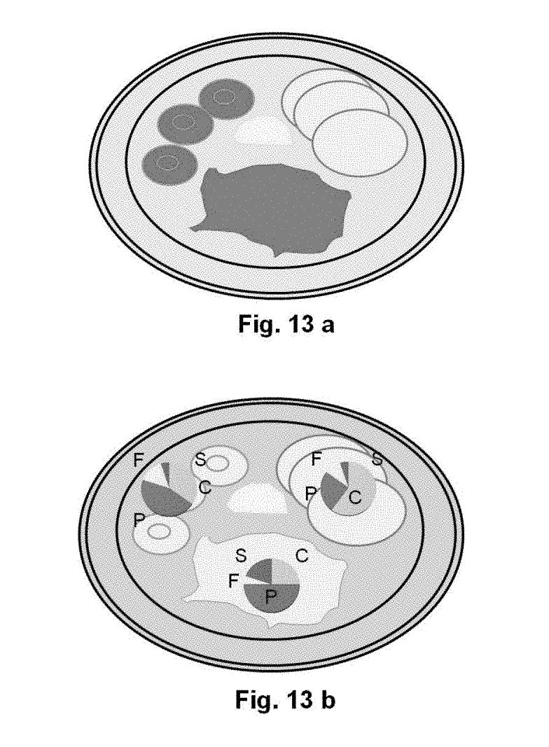

FIG. 13a illustrates a processed image of a plate with food items that have been color-coded based on their dominant nutritional attribute value depicting white for food items that are mostly carbohydrate, like rice, red for protein-dominant items or green for vegetable/fiber-dominant items.

FIG. 13b illustrates a processed image of a plate with food items on which are superimposed, pie-charts or other like representations indicating the relative proportions of various nutritional attributes of each food item such as, for example, a pie chart on a rice-based item showing a majority of carbohydrate ‘C’, followed by fiber ‘F’, protein ‘P’ and sodium ‘S’.

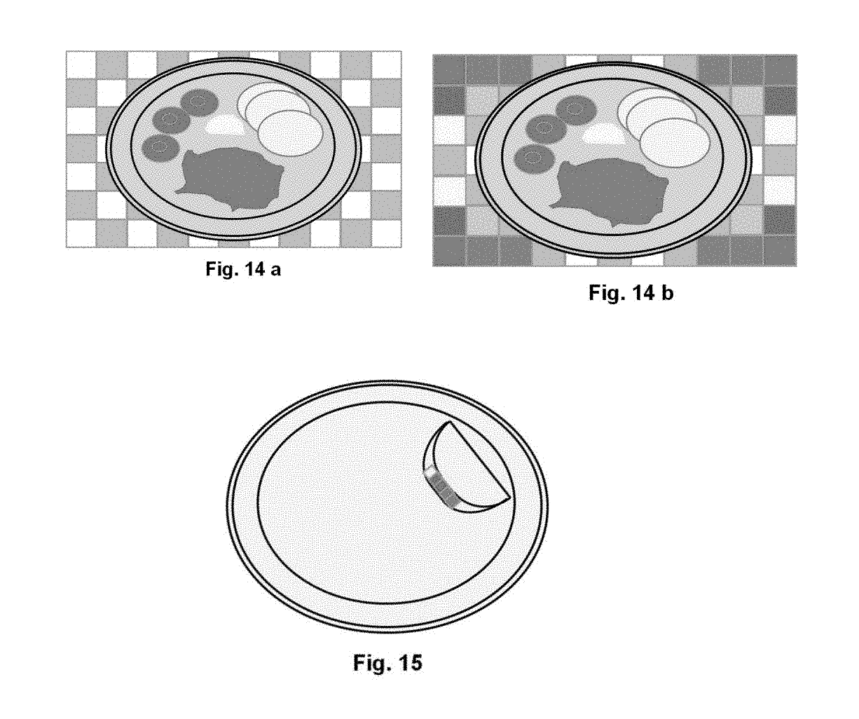

FIG. 14a a placemat printed with grey and white squares of one-inch or standard size to provide a sizing reference as an aid for estimating the size and/or quantity of the food items on a plate that is placed on top of the placemat.

FIG. 14b illustrates a placemat printed with colored squares of one-inch or standard size to provide color and sizing reference as an aid for identifying the food items and estimating their size and/or quantity on a plate that is placed on top of the placemat.

FIG. 15 shows a plate partition device shown in FIG. 12c printed with standard sized colored squares that serve as reference in identifying and estimating food items on the plate.

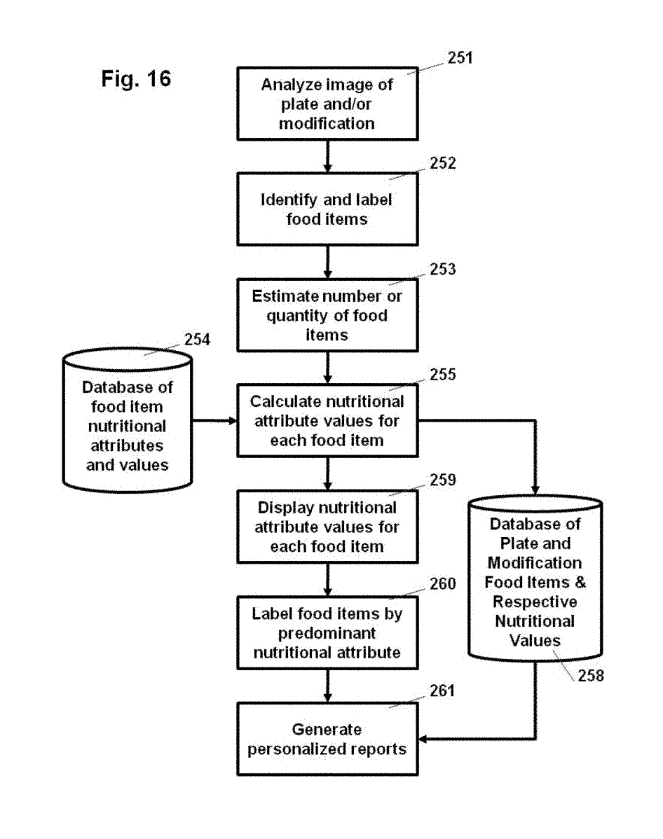

FIG. 16 illustrates the process flow for estimating the nutritional values of the food items for either a plate of food or the food items set aside or rejected as the modification, and report generation. The photograph of a plate of food or modification is analyzed [251] for individual food items, identified and labeled [252], and their respective number or quantities are estimated [253].The total nutritional attribute values such as carbohydrate, protein, fiber, etc. of each food item in the photograph are calculated [255], by taking the product of the quantity or number of a specific food item and its respective nutritional attribute values from a database [254] of such values for a large number of food items. The calculated values are displayed [259] in tabular form and each item is labeled by its predominant nutritional attribute [260]. These calculations are stored in a database [258], and personalized reports are generated [261] for individual users. These reports also depict past trends, current status and future predictions and display them at the time of analyzing.

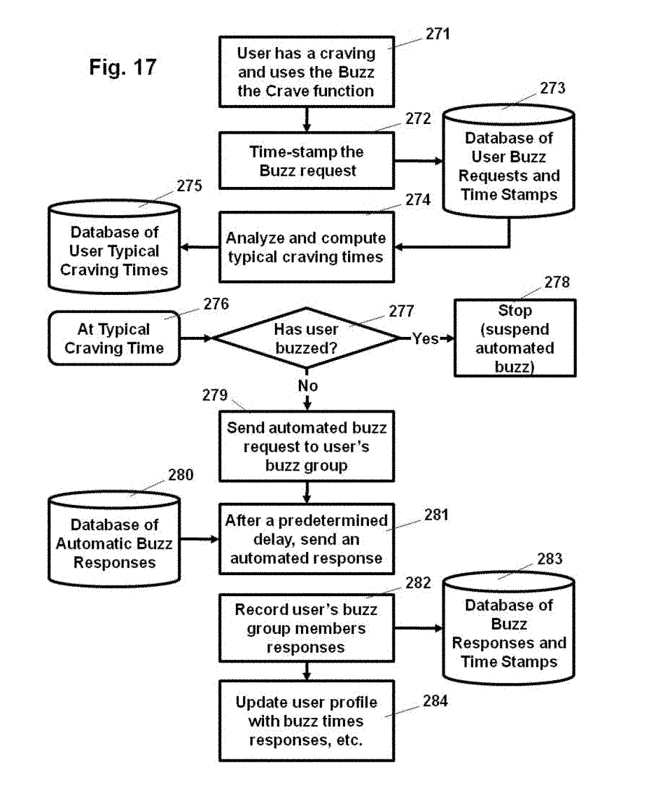

FIG. 17 illustrates the process flow for predicting a user's craving times and generating proactive distractions to it. Snacking adds unnecessary calories, carbs, sodium, fats etc., to the daily intake and can thwart weight-loss. A user may get a craving at some time of day for a snack; these cravings typically last several minutes and by distracting the person, attention is diverted and the craving passes. The ‘Buzz’ function is a means to request ‘distractions’ from friends or others. When a user has a craving and uses the ‘Buzz’ function [271], the use is time-stamped [272], and the buzz requests are stored in a database [273]. These requests are analyzed and typical craving times of day are computed [274], for that particular user and stored in a database [275].

At a typical craving time [276], the system checks if the user has already used the Buzz function [277]. If the user has not used the Buzz function, the system automatically sends a proactive Buzz request to the user's friends [279]. If the user has already used the Buzz function, no action is taken and any automated buzz is suspended [278]. After a predetermined delay, the system automatically sends a buzz response to the user [281], drawing from a database of automatic buzz responses [280]. All buzzes and responses are recorded [282] and stored in a database [283].

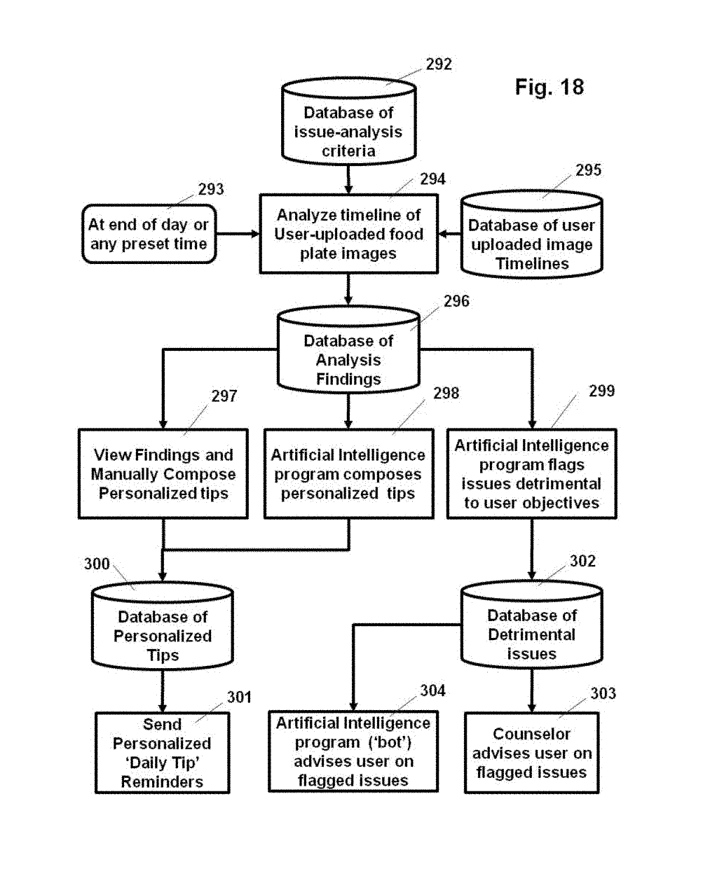

FIG. 18 illustrates the process flow for monitoring the user's eating patterns and flag issues raised by the user and providing personalized advice. At any preset time, e.g. end of day [293], the AI, nutritionists or other staff analyze the user-uploaded images [294], from the database of user-uploaded images [295], applying issue-analysis criteria [292]. The criteria may include ‘eating the same food items 3 days in a row’, ‘eating more than 5 servings of carbohydrates’, etc. The findings from the analysis are stored in a database [296]. A nutritionist or other staff professional views the findings for a particular user and manually composes personalized tips [297] that are stored in a database of personalized tips [300]. At some convenient time of day, the system automatically sends the personalized tips to the respective user [301].

Alternatively, an artificial intelligence (AI) program [298] may compose such personalized tips. An artificial intelligence program [299] also flags issues that may be detrimental to achievement of user objectives (such as eating too many calories, which would not help achieve a weight-loss objective) and store such issues in a database [302]. This database may be used by a counselor to provide advice to the user on flagged issues [303]. The artificial intelligence program [304] would use the database [302] to compose and send personalized messages to advise the user about their respective flagged issues.

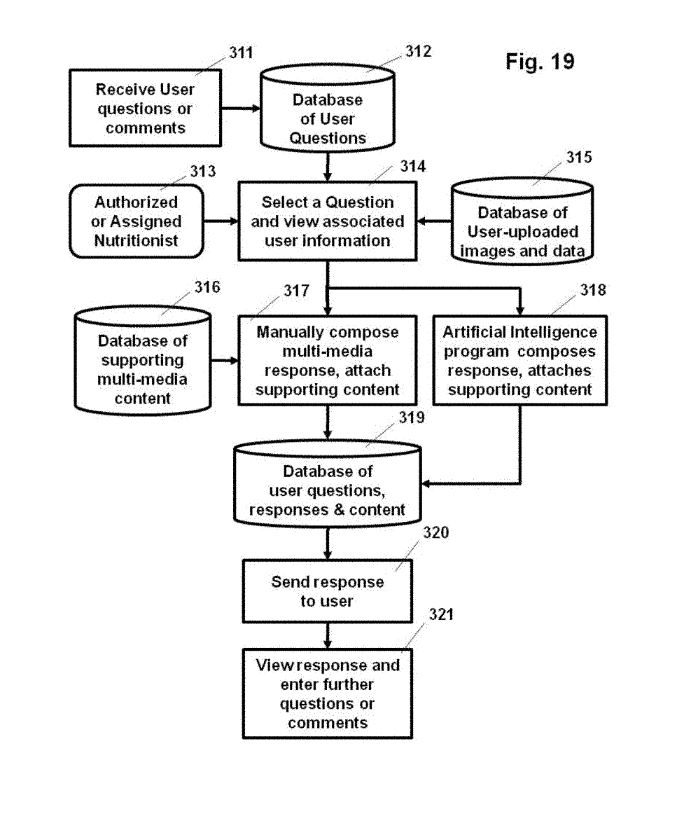

FIG. 19 illustrates the process flow for providing coaching to a user through two-way rich media. The registered user's questions are received [311] by the system and stored in a database [312]. An authorized or user-assigned nutritionist [313] selects a question and views associated user information [314], which may be in the forms of text, images, graphics, voice, etc. stored in a database [315]. Based on analysis of the questions and associated information, the nutritionist or other professional manually composes a multi-media response [317], selecting and attaching appropriate content from the database [316] and storing the response in a database [319]. Alternatively, an artificial intelligence program [318] may compose and store the response. The system sends the response to the user [320]. The user may view the response and enter further clarifying questions or comments [321]. Such two-way interactions continue until the user concludes the interaction.

Various modifications and adaptations on the described preferred embodiments can be configured without departing from the scope and spirit of the invention. Therefore, it is to be understood that within the scope of the appended claims, the invention may be practiced other than as specifically described herein.

We claim:

1. A method for providing real-time assistance to users in achieving their personalized health goals through a mobile phone, wherein the registered user obtains assistance in neutralizing a food craving comprising the steps of:
initiating a craving help request in dealing with food cravings at any time by the registered user;
setting up other users as helpers or groups along with their contact numbers and other information to whom the craving help request is sent;
specifying the helpers or groups to whom the request should be sent;
broadcasting the craving request to the specified helpers or groups and notify the registered users, helpers or groups individually;
responding and attempting to distract the requesting registered user from the craving by calling, texting, sending video clips, links or other means by the registered/unregistered helpers; and
maintaining the craving help request open for a specified duration and automatically closing it after receiving at least one response;
wherein the AI automatically responds to the craving request to distract the registered user from the craving at typical snack-craving times or at anytime or if there is no response from any helper or group after a set duration.

2. The method according to claim 1, wherein the AI automatically engages different friends when having different kinds of cravings by mapping specific cravings to a given friend's profile.

3. The method according to claim 1, wherein the AI automatically sends a reminder or a notification to a specially-designated friend to proactively distract a specific registered user from a craving.

4. The method according to claim 1, wherein the AI analyzes patterns of craving to predict the next time of day when a registered user might get a craving and proactively suggests or engages a friend to respond to the craving.
